(19) United States
(12) Patent Application Publication
(10) Pub. No.: US 2011/0020474 A1
(43) Pub. Date: Jan. 27, 2011

(54) Methods of Treating Infections of the Nail or Skin Using Hypohalite

(75) Inventors: Ramin Najafi, Novato, CA (US); Lu Wang, Moraga, CA (US)

Correspondence Address: Hamilton DeSanctis & Cha, LLP, 3239 El Camino Real, Suite 220, Palo Alto, CA, 94306, US

(73) Assignee: NovaBay Pharmaceuticals, Inc.

(21) Appl. No.: 12/843,676

(22) Filed: Jul. 26, 2010

Related U.S. Application Data
(60) Provisional application No. 61/228,917, filed on Jul. 27, 2009.

(51) Int. Cl. A61K 33/20; A61P 31/00; A61P 31/10

(52) U.S. Cl. 424/665

(57) ABSTRACT

The present application discloses a method of treating or preventing an infection of a nail, claw, hoof, or skin comprising administering a pharmaceutically acceptable formulation comprising hypohalite and a formulating agent to a subject in need thereof, and compositions thereof.

### RELATED APPLICATIONS

This application claims the benefit of U.S. Provisional Application No. 61/228,917, filed on Jul. 27, 2009, the entire contents of which are incorporated herein by reference.

### FIELD

This invention relates to methods of treating or preventing infections of the nail, claw, hoof or skin comprising administering a formulation of hypohalite to a subject in need thereof.

### BACKGROUND

Fungal infection of the nail, also referred to by the terms “onychomycosis” and “tinea unguium,” affect approximately 5% of the population worldwide, including 2-13% of the population of North America and Europe. Infection rates may be higher in subjects with HIV infection, diabetes, or who have suppressed immunological responses. Fungal infection of the nail are caused most commonly by dermatophytes, and less commonly by molds and yeasts, such as yeasts of the genus Candida. Onychomycosis can be categorized into several varieties, including distal and lateral subungual onychomycosis, endonyx onychomycosis, white superficial onychomycosis, proximal subungual onychomycosis, Candida onychomycosis, and total dystrophic onychomycosis. Nails may also be infected by certain bacteria or virus. Similar infections of the claw may affect animals such as cats, dogs, birds, and reptiles, and similar infections of the hoof may affect animals such as horses, cattle, goats, pigs and sheep.

Onychomycosis is presently treated primarily with oral antifungal agents. Oral antifungal agents include terbinafine (e.g. Lamisil®), itraconazole (e.g. Sporanox®), and fluconazole (e.g. Diflucan®). Such compounds can cause hepatic injury, however, and monitoring of liver enzymes may be required during treatment. Most topical agents such as Penlac® and Loceryl® tend to be less effective than the oral agents, except in mild cases that mainly affect the distal portion of the nail plate.

Sodium hypochlorite, commonly known as bleach, is known as a home remedy to treat onychomycosis. However, such treatments use bleach with no formulating agents, and thus do not provide for well-controlled, repeatable or convenient applications. Accordingly, success using such home remedies varies widely.

Other treatments of nail infections have either high risks of adverse effects or limited efficacy, in addition to other drawbacks such as drug interactions. Thus, there is a need for new treatments for onychomycosis and other infections of the nail, claw, hoof, or skin that are effective, safe and convenient to use.

### SUMMARY

This disclosure describes methods of treating or preventing infections of the nail, claw or hoof, comprising administering a pharmaceutically acceptable formulation of hypohalite and a formulating agent to a subject in need thereof.

In some embodiments, the infection is a fungal infection. In some embodiments, the hypohalite is hypochlorite.

In some embodiments, the formulating agent comprises a water-swellable polymer. For example, the water-swellable polymer can be a poly (ethylene oxide), or a polyacrylic acid.

In other embodiments, the formulating agent comprises a water-absorptive clay. For example, the water-absorptive clay can be bentonite.

In certain embodiments, the formulation has a pH from about 2 to about 12. In other embodiments, the formulation has a pH from about 6 to about 8 or from about 2 to about 6. In other embodiments, the formulation has a pH from about 3.5 to about 8.

This disclosure also describes a method of treating or preventing an infection of a nail, claw or hoof comprising administering to a subject in need thereof a pharmaceutically acceptable composition comprising a first active agent comprising hypohalite; a second active agent; and a formulating agent, wherein the second active agent is an antiinfective agent of a different class than the first active agent.

In certain embodiments, the second active agent is selected from the group consisting of allylamines, triazoles, imidazoles, amorolfine, ciclopirox, alamine, sodium pyrithione, bifonazole, propylene glycol, urea, lactic acid, benzyl alcohol, phenoxyethanol, phenethylalcohol, iodopropyl butyl carbamate, paraben, quaternary ammonium compounds, benzoyl peroxide, and chlorhexidine.

In other embodiments, the second active agent is an N-halogenated or N,N-dihalogenated amine compound. Examples of such compounds include, without limitation, 2-(dichloroamino)-2-methylpropane-1-sulfonic acid, 2-(chloroamino)-2-methylpropane-1-sulfonic acid, and N-chlorotaurine.

This disclosure also describes a bandage for the treatment or prevention of an infection of a nail, claw or hoof, comprising a nail-facing layer wherein the nail-facing layer contains a hypohalite formulation described herein. For example, the hypohalite formulation can comprise hypochlorite and a water-swellable polymer. The nail-facing layer of such a bandage may allow for release of hypohalite over a period of time. For example, a nail-facing layer can allow for release of hypohalite over about 24 to about 48 hours. The bandage may also include an adhesive stip. Similar to bandages, patches and boots (e.g. to fit over hooves of domestic or farm animals) may be used.

This disclosure also describes a method of treating or preventing infections of the skin comprising administering a pharmaceutically acceptable formulation of hypohalite and a formulating agent to a subject in need thereof. In certain embodiments, the skin infection is an uncomplicated skin and skin structure infection such as simple abscesses, cellulitis, folliculitis, furuncles, or impetiginous lesions.

The details of one or more embodiments are set forth in the accompanying figures and the description below. Other features, objects, and advantages will be apparent from the description and drawings, and from the claims.

### BRIEF DESCRIPTION OF THE DRAWINGS

FIG. 1A is a side view of a bandage having a nail-facing layer and an adhesive strip, and which is affixed to a peel sheet.

FIG. 1B is a top view of the bandage illustrated in FIG. 1A.

FIG. 1C shows the bandage illustrated in FIG. 1A partially peeled away from the peel sheet, exposing the nail-facing layer.

FIG. 2 shows an array of bandages of varying size affixed to a single peel sheet.

### DETAILED DESCRIPTION

As utilized in accordance with the present disclosure, the following terms, unless otherwise indicated, shall be understood to have the following meanings:

“Active agent” refers to a pharmaceutically active compound, for example an antifungal, antibacterial, or antiviral compound. Active agents include hypohalites.

The terms “composition,” “formulation,” and “solution” refer to a preparation comprising an active agent, e.g. hypohalite.

“Hypochlorite” refers to any compound or salt (e.g. a sodium, potassium, or calcium salt) that yields hypochlorite, i.e. ClO⁻. For clarity, the term “hypochlorite” encompasses hypochlorous acid (HOCl).

“Hypohalite” refers to any compound or salt (e.g. a sodium, potassium, or calcium salt) that yields hypohalite, i.e. an XO⁻ anion wherein X is a halogen, including hypochlorite (ClO⁻) and hypobromite (BrO⁻). For clarity, the term “hypohalite” encompasses hypochlorous acid (HOCl) and hypobromous acid (HOBr).

“Nail or nearby tissue” refers to tissues and structures of the nail, including the nail plate, nail bed, nail matrix, nail root, eponychium (cuticle), perinychium, hyponychium, and proximal and lateral nail folds, and corresponding structures of the claw. This term also refers to tissues and structures of the hoof, including the coronet, wall, toe, quarter, heel, bulb, frog and sole.

“Pharmaceutically acceptable” refers to that which is generally safe, non-toxic, and acceptable for veterinary as well as human pharmaceutical use.

“Prevent”, “preventing” and “prevention” of an infection refer to reducing the risk of a subject from developing an infection, or reducing the frequency or severity of an infection in a subject.

“Room temperature” refers to a temperature from about 7° C. to about 32° C., e.g. from about 18° C. to about 31° C.

“Salt” refers to a cation or anion coupled with an anion or a cation, either in solution or as a solid. Salts include pharmaceutically acceptable salts as well as solvent addition forms (solvates).

“Stable” and “stability” refer to the ability of a formulation described herein to remain suitable for its intended use.

“Subject” refers to any animal having a nail, claw, or hoof, including but not limited to a bird and reptile; an ungulate such as a horse, cow, bull (and other types of cattle), goat, pig, and sheep; a dog and cat, and other mammals such as a human.

The current disclosure relates to methods for treating or preventing infections of the nail, claw or hoof, comprising administering a pharmaceutically acceptable formulation comprising hypohalite and a formulating agent to a subject in need thereof. In certain embodiments, the infection can be a fungal infection. In certain embodiments, the infection may be a bacterial or viral infection. In certain embodiments, the subject can be a human.

In some embodiments, the hypohalite may be hypochlorite. In other embodiments, the hypohalite may be hypobromite.

Hypohalite may be derived or produced in several ways, as described in the following non-limiting examples.

For way of first example, sodium hypochlorite (bleach) is commercially available from such vendors as J. T. Baker. Hypohalite may also be derived from other sources known in the art, e.g. from trichloro isocyanuric acid, sodium or potassium dichloroisocyanurates, N-chloro- and N-bromoalkane sulphonamides, and similar agents (see, e.g. U. Tilstam and H. Weinmann Trichloroisocyanuric Acid: A Safe and Efficient Oxidant, Org. Proc. Res. Dev., 6(4), 384-393 (2002)). Hypochlorous acid may be produced by bubbling chlorine gas though water. Similar means may be used to produce other hypohalite solutions.

Solutions of sodium hypochlorite may be acidified using HCl. In this way a basic and caustic solution of sodium hypohalite may be brought to a more desirable (i.e. pharmaceutically acceptable) pH range.

Hypohalite may also be produced by electrolysis of water containing halide salt. During the electrolysis process, the halide salt is oxidized. In the case of NaCl, the chloride ions undergo an oxidative process which results in the production of chlorine gas, which in turn forms hypochlorous acid.

In another example of making a hypohalite solution, U.S. Pat. No. 6,426,066 describes a solution containing HOCl prepared by the electrolysis of a solution containing several salts. A salt mixture was prepared by adding 14.2 g of KCl (J. T. Baker), 8.05 g of MgCl₂, 6H₂O (J. T. Baker) and 235.5 g NaCl (non-iodated, Morton). This salt mixture was added to tap water at a concentration of 5.38 g/L of water to prepare a starting solution. About 2.5 L of solution was placed in a first compartment of an electrolyzer (e.g. a Suntron MWB-2 model electrolyzer), and about 2.5 L of solution was placed in a second electrolysis compartment. A power source (e.g. the electrolyzer) was turned on and power was applied for 15 minutes. The electrolysis was carried out at 25° C. to 30° C. with no external heat added and no heat removed.

In another example of making a hypohalite solution, U.S. Publication No. 2002/0160053 and U.S. Publication No. 2005/0196462 describe oxidative reductive potential water solutions containing free chlorine, which consists of HOCl, Off, and other chlorine species (e.g. Cr, 002 ClO₂, and Cl₂) known to be present in aqueous solutions containing such species depending on pH, temperature, and other conditions.

The hypohalite of the present disclosure may be in an aqueous solution, formulation, or composition. No matter what the source of or method of making the hypohalite, the corresponding acid and other ions may also be present. For example, in the case of hypochlorite, HOCl and Cl⁻ may also be present. The relative amounts of these constituents are greatly affected by the pH of the solution or composition. Referring again to hypochlorite, under acidic conditions, most of the hypohalite is in the form of HOCl. The relative concentrations of HOCl and OCl⁻ are roughly equal at a pH of about 7.5.

Concentrations of hypohalite may range from about 0.01% to about 6.0% (w/v). For example, in certain embodiments, the concentration of hypohalite may range from about 0.1% to about 2.0% (w/v).

The pH of the formulation may range from about 2 to about 12. In some embodiments the formulation can be acidic, e.g. with a pH from about 2 to about 6. In other embodiments the formulation can be generally neutral range (slightly acidic to slightly basic), with a pH from about 6 to about 8; or basic, with a pH from about 8 to about 12. In some embodiments, the pH may be from about 3.5 to about 8. For example, and without limitation, in embodiments wherein the hypohalite is hypochlorite (including hypochlorous acid) made in accordance with the methods described in U.S. Pat. No. 6,426,066 as above, the pH of the formulation may be from about 2 to about 6, e.g. from 2.2 to 4.5, e.g. from 2.4 to 3.5.

In some embodiments, the formulation comprising hypohalite and a formulating agent is stable. In certain embodiments, formulations are stable for 24 hours or more. In other embodiments, formulations are stable for about one month or more. In yet other embodiments, formulations are stable for about three months or more. Stability at a given time is typically a function of temperature and storage conditions (e.g. container material), among other factors. The amount of active agent in a formulation can decrease over time with the formulation still being considered “stable”, i.e. remaining suitable for its intended use. Whether a formulation is stable or not can be determined, among other factors, by its microbicidal (e.g. anti-fungal, anti-bacterial, or anti-viral) activity, and will depend on the particular intended use of that formulation.

In some embodiments, the formulation is selected from the group consisting of a cream, emulsion, film, gel, lacquer, lotion, ointment, paste, polish (e.g. nail polish), powder, spray, suspension and varnish, as well as solutions and gaseous formulations, such as aerosols. Such formulations may be prepared using standard techniques and an appropriate formulating agent. In other embodiments, the formulating agent is selected from the group consisting of a carrier, clay, excipient, film-forming agent, humectant, penetration enhancer, polymer, plasticizer, solvent, co-solvent, and surfactant. A combination of formulating agents described above may also be used.

In one aspect, the formulation of the present application is a gel. A gel formulation of hypohalite can be prepared by conventional pharmaceutical methods. For example, a gel formulation can comprise hypohalite and a gelling agent as the formulating agent.

The gelling agent can be a water-swellable polymer (that is, a polymer that hydrates in water to form viscous solutions or suspensions). Examples of water-swellable polymers include poly (ethylene oxide) polymers (e.g. Polyox®) and poly (acrylic acid) polymers (e.g. Carbopol®). Commercial grades of Polyox® include but are not limited to: WSR N-10, WSR N-80, WSR N-750, WSR N-3000, WSR-205, WSR-1105, WSR N-12K, WSR N-60K, WSR-301, WSR Coagulant, WSR-308 UCARFLOC Polymer 300, UCARFLOC Polymer 302, UCARFLOC Polymer 304 and UCARFLOC Polymer 309 available from Dow Chemical Company. Carbopol® homopolymers are polymers of acrylic acid crosslinked with allyl sucrose or allyl pentaerythritol. Carbopol® copolymers are polymers of acrylic acid and C₁₀-C₃₀ alkyl acrylate crosslinked with allyl pentaerythritol. Carbopol® interpolymers are a carbomer homopolymer or copolymer that contains a block copolymer of polyethylene glycol and a long chain alkyl acid ester. Commercial grades of carbopol include but not limited to: Carbopol® homopolymers, Carbopol® copolymers, Carbopol® interpolymers, Noveon® AA-1 Polycarbophil (“AA1” or “AA gel”), Noveon® CA-1 Polycarbophil (calcium neutralized), Noveon® CA-2 Polycarbophil (calcium neutralized) available from Noveon, Inc. (Cleveland, Ohio, USA), and other commercially available grades of Carbopol® and Polycarbophil.

The polymeric gel formulations described herein are generally prepared as follows: the polymer is hydrated slowly in purified water with or without common pharmaceutical excipients such as sodium chloride, salts and buffers. An aqueous hypohalite (e.g. hypochlorous acid) solution is then added. The solution is then mixed and adjusted to the desired pH using a suitable acid or base, e.g. HCl or NaOH. Water/oil emulsions, hydrating agents, wetting agents, penetration enhancers, humectants, solvents, co-solvents, surfactants, and the like, as described below, may also be used in polymeric gel formulations. Concentrations of polymer (e.g. Polyox® and Carbopol® of all types), may be from about 0.01% to about 75.0% (by total weight of the formulation). By way example but not limitation, for direct topical application to the nail, claw, or hoof, a polymer concentration from about 0.01% to about 10%, e.g. about 0.1% to about 5%, may be used, whereas for methods using a patch, bandage, or boot, a polymer concentration of up to about 75% may be used.

In another aspect, the formulation of the present application is a paste. In these formulations, the formulating agent may be a clay, e.g. a water-swellable clay. For example, a paste formulation can comprise hypohalite and a synthetic clay. The synthetic clay can be a magnesium silicate having partial replacement of magnesium ions with lithium ions, e.g. about 5% to about 15% of the magnesium ions are replaced by lithium ions. The silicate structures of these clays are equivalent to that of the natural Hectorite. These compositions will have a paste-like consistency when present in an aqueous solution of hypohalite from about 3 percent to about 8 percent by weight. As with the gel formulation, hydrating agents, wetting agents, penetration enhancers, humectants, solvents, co-solvents, surfactants, and the like, may be used in clay formulations. The pH of such compositions may vary depending on the desired application. In certain applications, the compositions may be acidic, with a pH from about 2 to about 6; neutral, with a pH of about 6 to about 8; or basic, with a pH of about 8 to about 12. Another suitable water-swellable clay is VEEGUM® magnesium aluminum silicate (R. T. Vanderbilt). Additional suitable clays include other montmorillonite, bentonite, beidellite, hectorite, saponite, and stevensite, and their synthetic analogs.

Formulations may comprise a solvent or co-solvent. Suitable solvents and co-solvents include water, ethanol, acetone, methylacetate, ethyl acetate, butyl acetate, alkylmethyl sulfoxides (e.g. dimethyl sulfoxide), 2-propanol, methyl isobutyl ketone, 1-butanol, dichloromethane, and mixtures thereof. For example, a volatile organic solvent may be used so that a dry film containing the active agent forms over the nail after administration of the formulation. Formulations may also include pharmaceutically acceptable excipients which can be found in Remington: The Science and Practice of Pharmacy, R. Hendrickson, ed., 21st edition, Lippincott, Williams & Wilkins, Philadelphia, Pa., (2005) at pages 317-318, which is hereby incorporated herein by reference.

Formulations may also include a penetration enhancer. The penetration enhancers of the compositions and methods described herein can enhance penetration of the active agent into the nail and nearby tissue. Penetration enhancers include ethanol, propylene glycol, glycerol, ethyl laurate, isopropyl palmitate, isopropyl myristate, laurocapram (AZONE), dioxolanes, macrocyclic ketones, 1-decyl-thiolthyl-2-pyrrolidone (HP-101), oxazolidones and biodegradable penetration enhancers such as alkyl-2-(N,N-disubstituted amino) alkanoates (e.g., dodecyl-2-(N,N-dimethylamino) isopropionate (DDAIP)), N,N-disubstituted amino alkanol alkanoates) and mixtures thereof.

The amount of the penetration enhancer can be varied depending on the desired release rate and the specific active agent used. Generally, the penetration enhancer can be present in an amount ranging from about 0.1 weight percent to about 25 weight percent, based on the total weight of the formulation. In other aspects, the penetration enhancer can be present in an amount ranging from about 0.1 weight percent to about 10 weight percent, e.g. from about 0.5 weight percent to about 5 weight percent of the formulation.

In general, suitable penetration enhancers include the following classes of compounds: aliphatic and aromatic alcohols, sulfoxides, fatty alcohols, fatty acids, fatty acid esters, polyols, amides, surfactants, terpenes, alkanones, organic acids and mixtures thereof.

Suitable alcohols include, without limitation, ethanol, propanol, butanol, pentanol, hexanol, octanol, nonanol, decanol, 2-butanol, 2-pentanol, benzyl alcohol, phenoxyethanol, caprylic alcohol, decyl alcohol, lauryl alcohol, 2-lauryl alcohol, myristyl alcohol, cetyl alcohol, stearyl alcohol, oleyl alcohol, linolyl alcohol, linolenyl alcohol and mixtures thereof.

Volatile aliphatic alcohols having 2 to about 5 carbon atoms can provide a dual function of serving both as a volatile solvent and a penetration enhancer. Aromatic alcohols, such as benzyl alcohol, phenoxyethanol, and the like can provide a dual function of serving both as a substantially non-volatile, permeation enhancer and an auxiliary anti-infective. In certain aspects, suitable penetration enhancers are ethanol and benzyl alcohol.

Suitable sulfoxides include dimethylsulfoxide (DMSO), decylmethylsulfoxide, and mixtures thereof.

Suitable fatty acids include valeric, heptanoic, pelargonic, caproic, capric, lauric, myristic, stearic, oleic, linoleic, linolenic, caprylic, isovaleric, neopentanoic, neoheptanoic, neononanoic, trimethyl hexanoic, neodecanoic and isostearic acids, and mixtures thereof.

Suitable fatty acid esters include isopropyl n-butyrate, isopropyl n-hexanoate, isopropyl n-decanoate, isopropyl myristate, isopropyl palmitate, octyldodecyl myristate, ethyl acetate, butyl acetate, methyl acetate, methylvalerate, methylpropionate, diethyl sebacate, ethyl oleate, ethyl laurate, sucrose monolaurate, and mixtures thereof. Suitable polyols include propylene glycol, polyethylene glycol, ethylene glycol, diethylene glycol, triethylene glycol, dipropylene glycol, glycerol, propanediol, sorbitol, dextrans, butanediol, pentanediol, hexanetriol, and mixtures thereof.

Suitable amides include urea, dimethylacetamide, diethyltoluamide, dimethylformamide, dimethyloctamide, dimethyldecamide, pyrrolidone derivatives, 1-alkyl-4-imidazolin-2-one, cyclic amides, hexamethylenelauramide and its derivatives and mixtures thereof.

Suitable pyrrolidone derivatives include 1-methyl-2-pyrrolidone, 2-pyrrolidone, 1-lauryl-2-pyrrolidone, 1-lauryl-4-carboxy-2-pyrrolidone, 1-methyl-4-carboxy-2-pyrrolidone, 1-hexyl-4-carboxy-2-pyrrolidone, 1-decylthioethyl-2-pyrrolidone (HP-101), N-cyclohexylpyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-dimethylaminopropylpyrrolidone, N-cocoylpyrrolidone, N-tallowylpyrrolidone, fatty acid esters of N-(2-hydroxymethyl)-2-pyrrolidone, and mixtures thereof.

Suitable cyclic amides include, 1-dodecylazacycloheptan-2-one (laurocapram, AZONE), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyloctyl)azacycloheptan-2-one, 1-geranylazacyclohexan-2-one, 1-geranylazacyclopentan-2,5-dione, 1-farnesylazacyclopentan-2-one, and mixtures thereof.

Suitable surfactants include anionic surfactants, cationic surfactants, nonionic surfactants, amphoteric surfactants, bile salts and lecithin.

Suitable anionic surfactants include sodium laurate, sodium lauryl sulfate, and mixtures thereof. Suitable cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride, and mixtures thereof.

Suitable nonionic surfactants include alpha-hydro-omega-hydroxypoly(oxyethylene)-poly(oxypropyl) poly(oxyethylene) block copolymers, polyoxyethylene ethers, polyoxyethylene sorbitan esters, polyethylene glycol esters of fatty alcohols, and mixtures thereof. Suitable alpha-hydro-omega-hydroxy-poly(oxyethylene)-poly(oxypropyl) poly(oxyethylene) block copolymers include Poloxamers 182, 184, 231, and mixtures thereof. Suitable polyoxyethylene ethers include PEG-4 lauryl ether (BRIJ 30), PEG-2 oleyl ether (BRIJ 93), PEG-10 oleyl ether (BRIJ 96), PEG-20 oleyl ether (BRIJ 99), and mixtures thereof. Suitable polyoxyethylene sorbitan esters include the monolaurate (TWEEN 20) the monopalmitate (TWEEN 40), the monostearate (TWEEN 60), the monooleate (TWEEN 80), and mixtures thereof. Suitable polyethylene glycol esters of fatty acids include polyoxyethylene (8) monostearate (MYRJ 45), polyoxyethylene (30) monostearate (MYRJ 51), the polyoxyethylene (40) monostearate (MYRJ 52), and mixtures thereof. Saccharide surfactants such as dodecylmaltside may also be used.

Suitable amphoteric surfactants include, without limitation thereto, lauramidopropyl betaine, cocamidopropyl betaine, lauryl betaine, cocobetaine, cocamidopropylhydroxysultaine, aminopropyl laurylglutamide, sodium cocoamphoacetate, sodium lauroamphoacetate, disodium lauroamphodiacetate, disodium cocoamphodiacetate, sodium cocoamphopropionate, disodium lauroamphodipropionate, disodium cocoamphodipropionate, sodium lauriminodipropionate, disodium cocoamphocarboxymethylhydroxypropylsulfate, and the like.

Suitable bile salts include sodium cholate, sodium salts of laurocholic, glycolic and desoxycholic acids, and mixtures thereof.

Suitable terpenes include D-limonene, alpha-pinene, beta-enrene, alpha-terpineol, terpinen-4-ol, carvol, carvone, pulegone, piperitone, menthone, menthol, geraniol, cyclohexene oxide, limonene oxide, alpha-pinene oxide, cyclopentene oxide, 1,8-cineole, ylang ylang oil, anise oil, chenopodium oil, eucalyptus oil, and mixtures thereof.

Suitable organic acids include citric acid, succinic acid, salicylic acid, salicylates (including the methyl, ethyl and propyl glycol derivatives), tartaric acid, and mixtures thereof.

A penetration enhancer may also comprise an N,N-di(C₁-C₈) alkylamino substituted, (C₄-C₁₈) alkyl (C₂-C₁₈) carboxylic ester or pharmaceutically acceptable acid addition salt thereof. As used herein, the term (C₄-C₁₈) alkyl (C₂-C₁₈) carboxylic ester means an ester of a (C₄-C₁₈) alcohol and a (C₂-C₁₈) carboxylic acid. The term N,N-di(C₁-C₈) alkylamino substituted, in reference to a (C₄-C₁₈) alkyl (C₂-C₁₈) carboxylic ester means that either the alcohol portion or the carboxylic acid portion from which the ester is prepared bears an amino substituent NRₓR_{y}, wherein Rₓ and R_{y} are each independently a (C₁-C₈) alkyl group; in certain aspects, they are both methyl groups. Examples of such penetration enhancers include dodecyl-2-(N,N-dimethylamino) propionate (DDAIP); dodecyl-2-(N,N-dimethylamino)-acetate (DDAA); 1-(N,N-dimethylamino)-2-propyl dodecanoate (DAIPD); 1-(N,N-dimethylamino)-2-propyl myristate (DAIPM); 1-(N,N-dimethylamino)-2-propyl oleate (DAIPO); and pharmaceutically acceptable acid addition salts thereof.

In certain aspects, the penetration enhancer useful in the formulations of the present application is DDAIP, alone or in combination with an auxiliary permeation enhancer. DDAIP HCl is available from Steroids, Ltd. (Chicago, Ill.) and Pisgah Laboratories (Pisgah Forest, N.C.).

The formulations described herein may include humectants, which act as hygroscopic agents, increasing the amount of water absorbed, held and retained in the compositions of the application. Suitable humectants for the formulations described herein include but are not limited to acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof.

In certain aspects of the application, the humectant is present in the composition at concentrations from about 0.5 to about 40 percent by weight, e.g. from about 0.5 to about 20 percent by weight, e.g. from about 0.5 to about 12 percent by weight.

The formulations described herein may also include a film-forming agent. The film-forming agent may be a film-forming polymer comprising a vinylpyrrolidone monomer unit, including a homopolymer, (i.e., polyvinylpyrrolidone), a copolymer and a complex thereof, a gum, a resin, or the like. The term copolymer as used herein means any polymer comprising two or more different monomer repeating units and includes polymers commonly referred to as terpolymers, tetrapolymers and the like. Other film-forming agents, such as aluminium phyllosilicates (e.g. bentonite) and similar film-forming clays, may also be used.

Exemplary film-forming polymers containing vinylpyrrolidone (VP) monomer units, are polyvinylpyrrolidone (PVP), sold in a range of viscosity grades, and varying weight average molecular weights in the range of about 8,000 to about 3,000,000 Daltons

(PVP K homopolymer series). PVP is sold under the trade name KOLLIDON CL by BASF Corporation. A USP grade of povidone (PVP) is one such film forming agent.

Exemplary film-forming copolymers include vinylpyrrolidone/vinylacetate (VA) copolymers available in a range of mole ratios of VP/VA such as the PVPNVA copolymer series sold by ISP, and the like. In another aspect, the polymer is a polyvinylpyrrolidone having a weight average molecular weight in the range of about 45,000-60,000 Daltons. Film-forming polymer can also be naturally occurring or modified polymers such as cellulose derivatives.

Exemplary gums include agar gum, carrageenan gum, ghati gum, karaya gum, rhamson gum, xanthan gum and the like. Exemplary resins include carbomers (as described above), including CARBOPOL 940. Other polyacrylic acid polymers suitable for use are those commercially available under the designation PEMULEN (Noveon Inc.). The PEMULEN (The Lubrizol Corporation) polymers are copolymers of C₁₀ to C₃₀ alkyl acrylates and one or more monomers of acrylic acid, methacrylic acid or one of their simple esters cross-linked with an allyl ether of sucrose or an allyl ether of pentaerythritol. POLYCARBOPHIL (A. H. Robbins Company, Inc.) is another useful polyacrylic acid polymer, which is cross-linked with divinyl glycol.

There is no limitation on the form (i.e., liquid or powder) of the hydrophilic film-forming polymer used, or the amount used, as long as, the nail coat composition can be applied to the nail and forms a film thereon.

For example, most nail polishes are made of nitrocellulose dissolved in a solvent (e.g. butyl acetate or ethyl acetate) and either left clear or colored with various pigments. Basic components included are: film forming agents, resins and plasticizers, solvents, and coloring agents. Adhesive polymers (e.g. tosylamide-formaldehyde resin) ensure the nitrocellulose adheres to the nail's surface. Plasticizers (e.g. camphor) are chemicals that link between polymer chains, spacing them to make the film sufficiently flexible after drying. Pigments and sparkling particles (e.g. mica) add desired color and reflecting characteristics. Thickening agents (e.g. stearalkonium hectorite) are added to maintain the sparkling particles in suspension while in the bottle. Ultraviolet stabilizers (e.g. benozophenone-1) resist color changes when the dry film is exposed to direct sunlight.

One of skill in the art, upon reading this disclosure will appreciate that any one formulating agent listed above can be useful in more than one manner in the formulations of the present application. That is, for example, the same formulating agent can be a volatile solvent and a penetration enhancer, and so on.

The current disclosure relates to any appropriate means for administering the composition to the subject. For example, the compositions may be applied with a pipette or eye-dropper, or a cotton or fabric applicator.

In certain applications, it may be desirable to administer the composition continually over a certain period of time. Therefore, this disclosure also relates to a device or apparatus for treating or preventing infections of the nail, claw, or hoof, wherein the device or apparatus enables application of hypohalite to the nail or nearby tissue of a subject in need thereof. In some embodiments, a device can enable application of hypohalite mainly to the outer (dorsal) surface of the nail (the hypohalite, once applied, may then penetrate through the nail, e.g. to the nail bed). Such apparatus may be a bandage, wound dressing or similar device.

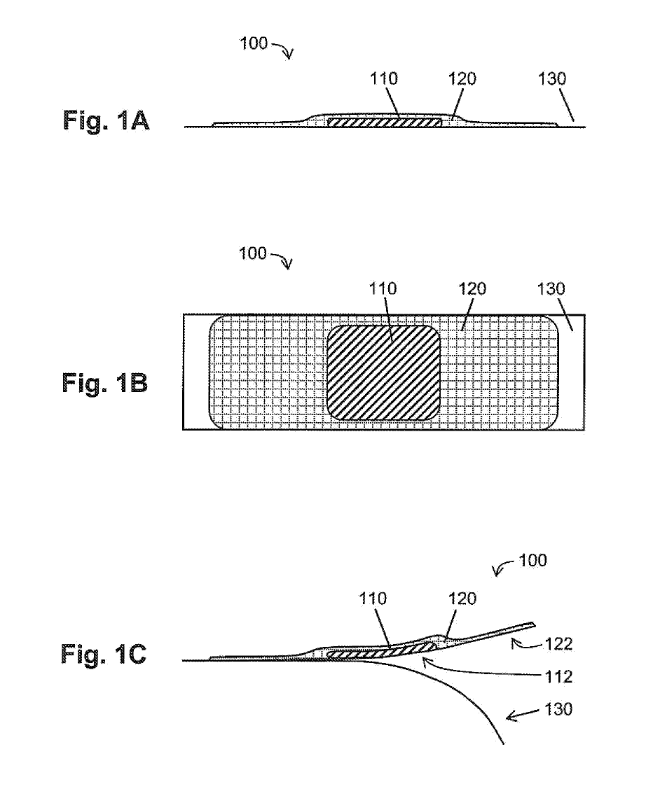

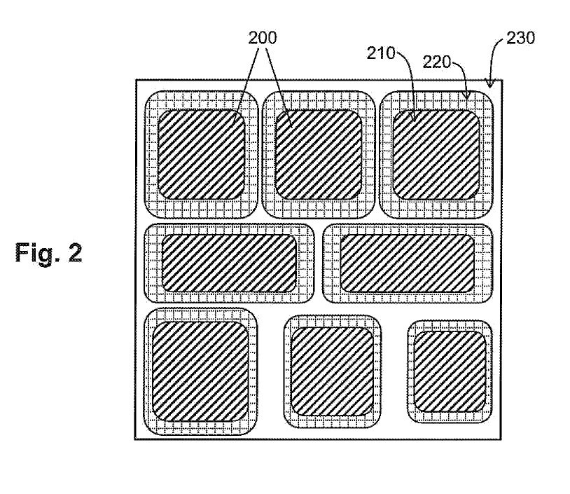

Examples of bandages are illustrated in FIGS. 1 and 2. Referring to FIGS. 1A (side view) and 1B (top view), bandage 100 comprises a nail-facing layer 110 and adhesive strip 120, and can be removably affixed to peel sheet 130. Nail facing layer 110 and adhesive strip 120 are flexible and can form a single piece. Nail facing layer 110 contains an active agent, e.g. hypohalite, in a formulation or composition described herein. When bandage 100 is applied to a subject, nail-facing layer 110 allows the active agent to contact the nail or nearby tissue. Nail-facing layer 110 can also include a polymer, gel, ointment, or other formulating agent described herein that allows it to conform to the nail or surrounding tissue when applied, and to be removed easily without causing pain or damage to the underlying tissue. Nail facing layer 110 may or may not be adhesive. Adhesive strip 120 may be formed from woven or nonwoven fabrics, plastic films, laminates thereof, and other materials known in the art. Adhesive strip 120 may also comprise an adhesive surface that allows the bandage to be reversibly secured to the subject. Suitable adhesives are well known in the art, and include adhesives based on acetates (e.g. vinyl acetates), acrylic acid (e.g. Duro-Tak 387-2516 by National Starch). Alternatively, acrylates, polyesters, polystyrenes, polyurethanes, silicones, styrene-isoprene-styrene block copolymers, and mixtures thereof may also be used. For example, the silicone polymer BIO PSA 7-4102, a polymer in solution in ethyl acetate supplied by Dow Corning, may be used. Bandage 100 is similar to a typical adhesive bandage (e.g. Band-Aid®, Curad®, etc.) in other respects. For example, adhesive strip 120 may be “skin-friendly” (removable without undue pain or discomfort) and may be perforated to enable perspiration to escape.

FIG. 1C shows bandage 100 (side view) partially peeled away from peel sheet 130, e.g. before being applied to a subject. This illustration shows the ventral sides 112 and 122 of nail-facing layer 110 and adhesive strip 120, respectively, which contact the subject when applied.

Referring to FIG. 2, bandages 200 comprise nail-facing layers 210 and adhesive strips 220, and are removably affixed to peel sheet 230. In this example, several bandages are affixed to one peel sheet, and are available in different sizes, e.g. to conform to different nail sizes.

Bandages may allow for release of active agents, such as hypohalite, over a certain period of time. For example, a bandage can allow hypohalite to be released over a period of about 12 hours, or over a period from about 24 to about 48 hours. Such bandages could be applied to subjects accordingly, e.g. about twice per day to about once every two days, to allow convenient and continual application of active agent to the nail and surrounding tissue. Bandages with other periods of release of active agent may be easily envisaged.

In another aspect, the method of the present application comprises application of hypohalite to the nail periungually or subungually. These methods can employ any suitable hypohalite formulation. For instance, the formulating agent can comprise a biodegradable pharmaceutically acceptable thermoplastic polymer that is at least substantially insoluble in aqueous medium or body fluid, a pharmaceutically acceptable biocompatible solvent that is water soluble, and a therapeutically effective amount of an active ingredient, wherein the thermoplastic polymer and biocompatible solvent are present in concentrations effective to form the implant in situ. The active ingredient may be miscible in the polymer and/or solvent to provide a homogeneous mixture with the polymer, or insoluble to varying degrees in the polymer and/or solvent to form a suspension or dispersion with the polymer.

The implants are solid articles and may include microcapsules, microparticles, structured articles such as sutures, staples, medical devices, stents and the like as well as monolithic implants and implant films, filamentous membranes and matrices. In certain aspects, the implant devices are biodegradable.

In certain aspects, the formulations (and bandages and implants comprising the same) are controlled-release formulations, i.e. formulations that release an active agent, e.g. hypohalite, over a period of time, e.g. from about 24 to about 48 hours. Such controlled release can be over a longer period of time, e.g. over about 14 days, over at least one month, or more. The composition of a suitable controlled-release composition may be tailored according to the release time required, for example the release period of the implanted composition being about one to about six months. In another aspect, the composition comprises a biodegradable polymer (poly-lactide co-glycolide) based delivery system and the polymer composition is selected to provide a release time of about one to about six months.

In certain aspects, where even longer term treatment is required, for example in onychomycosis, continuous dosing with an active agent may be necessary over extended periods of time, for example nine months, twelve months, or more. For such treatment, compositions and implants with a release period of one month, two months, three months, or six months, are prepared and used in the treatment repeatedly as required. The use of such extended release compositions can result in reduction in overall pain and discomfort to the subject during the treatment period, as well as increased compliance with treatment regimes. In an aspect, the composition or implant is biodegradable thus obviating the need for surgical procedures to remove the composition or implant. In another aspect, the compositions and implants are placed subdermally and sufficiently near the nail, or subungually, so as to afford accumulation of the active ingredient in and around the nail while at the same time minimizing systemic exposure.

In one aspect, the flowable compositions of the present application comprise an Atrigel biodegradable polymer (poly-lactide co-glycolide) based delivery system, wherein the polymers are dissolved in a biocompatible solvent. In other aspects, the polymers for use in the flowable compositions comprise biodegradable polymer (poly-lactide co-glycolide) based delivery systems, the blend ratio of monomers being generally about 90/10 to 10/90 (by weight) and about 25/75 through about 75/25. In other aspects, the biodegradable polymer is selected from 75/25 PLG; 85/15 PLG; 85/15 PLGH or 80/20 PLGH. Suitable biodegradable polymers for use in the compositions of the present application are those that afford release of the specific active agent over the intended period of time in situ.

Solvents suitable for use in the flowable composition are biocompatible and are at least slightly soluble in aqueous medium, body fluid, or water. The organic solvent is at least moderately soluble in aqueous medium, body fluid, or water. Testing methods to select such suitable biodegradable polymers and biocompatible solvents for use in accordance with the present application with an active ingredient are well-known to persons of skill in the art.

The flowable composition is suitable for injection under the nails of a subject where it forms a pharmaceutically acceptable solid matrix. In one aspect of the flowable composition, a biologically active agent is included and the solid implant will release the active agent at a controlled rate. The rate of release may be altered to be faster or slower by inclusion of a rate-modifying agent that is well known in the art.

When a subject is suffering from a disease of the nail that results in separation of the nail plate from the nail bed, such as in certain forms of onychomycosis, insufficient tissue fluid may be present to afford formation of the flowable composition into a solid matrix. In such cases, the flowable composition is deposited topically in the subungual space between the nail plate and nail bed and a suitable amount of aqueous solution is introduced in a suitable manner to the composition, either simultaneously or sequentially, so as to afford formation of the solid matrix implant.

In one aspect, the present application provides injectable nanoparticulate formulations of hypohalite that can comprise high drug (or active agent) concentrations in low injection volumes, with durations of action that can be controlled to give efficacious blood levels through manipulation of particle size and hence dissolution for periods of about one week or greater. Such composition of the application are administered via injection, such as by intramuscular or subcutaneously, to form a drug depot. The drug depot results in efficacious levels of drug up to about one week or greater. Thus, in certain aspects, the application provides compositions comprising nanoparticulate particles comprising hypohalite and at least one surface stabilizer.

The surface stabilizers are adsorbed to or associated with the surface of the nanoparticulate particles. Surface stabilizers useful herein do not chemically react with the nanoparticulate formulations. Individual molecules of the surface stabilizer are essentially free of intermolecular cross-linkages. The compositions can comprise two or more surface stabilizers.

Representative examples of useful surface stabilizers include but are not limited to low viscosity hydroxypropyl cellulose (HPC or HPC-SL); hydroxypropyl methyl cellulose (HPMC); hydroxymethyl cellulose (HMC); ethycellulose; povidone; Pluronics; sodium deoxycholate; PEG-Phospholipids; Tyloxapol and other approved tritons, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens such as e.g., Tween 20 and Tween 80 (ICI Speciality Chemicals)); polyethylene glycols (e.g., Carbowaxs 3550 and 934 (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68 and F108, which are block copolymers of ethylene oxide and propylene oxide); poloxamines (e.g., Tetronic 908, also known as Poloxamine 908, which is a tetrafunctional block copolymer derived from sequential addition of propylene oxide and ethylene oxide to ethylenediamine (BASF Wyandotte Corporation, Parsippany, N.J.)); Tetronic 1508 (T-1508) (BASF Wyandotte Corporation), Tritons X-200′, which is an alkyl aryl polyether sulfonate (Rohm and Haas); Crodestas F-110, which is a mixture of sucrose stearate and sucrose distearate (Croda Inc.); p-isononylphenoxypoly-(glycidol), also known as Surfactant 10-G (Olin Chemicals, Stamford, Conn.); Crodestas SL-40 (Croda, Inc.); and SA90HCO (Eastman Kodak Co.); decanoyl-N-methylglucamide; n-decyl beta-D-glucopyranoside; n-decyl beta-D-maltopyranoside; n-dodecyl beta-D-glucopyranoside; n-dodecyl beta-D-maltoside; heptanoyl-N-methylglucamide; n-heptyl-beta-D-glucopyranoside; n-heptyl beta-D-thioglucoside; n-hexyl beta-D-glucopyranoside; nonanoyl-N-methylglucamide; n-nonyl beta-D-glucopyranoside; octanoyl-N-methylglucamide; n-octyl-beta-D-glucopyranoside; octyl beta-D-thioglucopyranoside; PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like.

In other aspects of the application, the injectable nanoparticulate formulations of the present application are injected subungually or periungually for the treatment of onychomycosis. In certain other aspects, the injectable nanoparticulate formulations of the present application are injected under skin which is near a nail.

The current disclosure also relates to methods for treating or preventing infections of the nail, claw, or hoof, comprising administering to a subject in need thereof a composition or formulation comprising (i) a first active agent comprising hypohalite; and (ii) a second active agent, wherein the second active agent is an antiinfective agent of a different class than the first active agent. In these embodiments, the first active agent can be distinguished from the second active agent in that the first active agent is an acid or inorganic salt of a hypohalite (e.g. HOCl or NaOCl), the second active agent is not.

Such second active agents include, but are not limited to oral antifungal agents such as terbinafine, itraconazole, and fluconazole, as well as topical antifungal agents such as allylamines (e.g. terbinafine), triazoles (e.g. itraconazole and fluconazole), imidazoles (e.g. ketoconazole, miconazole, clotrimazole, and econazole), amorolfine, ciclopirox, alamine, sodium pyrithione, bifonazole plus urea, and propylene glycol plus urea plus lactic acid. Oral and topical antibacterial, antiviral, and other anti-infective agents may also be used. Such agents include, but are not limited to, benzyl alcohol, phenoxyethanol, phenethylalcohol, iodopropyl butyl carbamate, paraben, quaternary ammonium compounds (e.g., benzalkonium chloride), benzoyl peroxide, and chlorhexidine.

Further examples of second active agents include, without limitation, 2-(dichloroamino)-2-methylpropane-1-sulfonic acid, 2-(chloroamino)-2-methylpropane-1-sulfonic, and N-chlorotaurine. Other examples of suitable second active agents may be found in U.S. Pat. No. 7,462,361, U.S. Publication No. 2006/0247209, U.S. Publication No. 2010/0137349, and U.S. Publication No. 2010/0158818, the entire contents of each of which are incorporated herein by reference.

Formulations of the present invention may also be used in methods of treating or preventing skin infections, including complicated or uncomplicated skin and skin structure infections. Uncomplicated skin and skin structure infections include, by way of nonlimiting example, simple abscesses, cellulitis, folliculitis, furuncles, and impetiginous lesions. Complicated skin infections include infections either involving deeper soft tissue or requiring significant surgical intervention, such as infected ulcers, burns, and major abscesses or a significant underlying disease state that complicates the response to treatment. The skin infections may be viral, bacterial, or fungal in nature.

In one aspect, a method for the treatment or prevention of uncomplicated skin and skin structure infections comprises administering a composition comprising a hypohalite and a formulating agent to a subject in need thereof. The hypohalite can by hypochlorite. The formulating agent can be any of the formulating agents listed above, e.g. a water-swellable polymer or a water-absorptive clay. One method, for example, comprises administering a formulation comprising hypochlorite and a formulating agent comprising a poly (acrylic acid) to treat an uncomplicated skin and skin structure infection. The pH of this formulation may be from about 2 to about 12, for example from about 3.5 to about 8. This polymeric gel formulation may be applied topically to an infected site on a subject. For example, a formulation comprising hypohalite and a poly (acrylic acid) may be applied to treat simple abscesses, cellulitis, folliculitis, furuncles, or impetiginous lesions.

Similar to the bandages and patches for the nail mentioned above, a bandage or patch for the treatment or prevention of skin infections can comprise a skin-facing layer wherein the skin-facing layer comprises hypohalite and a formulating agent. For example, a patch can be used to treat a complicated or uncomplicated skin infection wherein the patch comprises hypochlorite and a water-swellable polymer.

In certain methods, the formulations for the treatment or prevention of skin infections can include a second active agent, as described above.

Other aspects of the current disclosure are set forth in the examples below. These examples are illustrative, and are not intended to limit the scope of the invention.

### EXAMPLES

### Example 1

### Paste Formulation

An exemplary composition of a synthetic clay in which about 10% of the magnesium ions are replaced by lithium ions is as follows (amounts reported as dry weight basis):

| Ingredient | Amount |
|---|---|
| SiO₂ | 60.4% |
| MgO | 26.0% |
| Li₂O | 1.1% |
| Na₂O | 3.0% |
| Fe₂O₃ | 0.02% |
| CaO | 0.20% |
| SO₃ | 0.10% |
| CO₂ | 0.29% |
| H₂O | 6.9% |

Such a formulation may be prepared as follows: about 2000 grams of a buffer solution is prepared from 2.1 grams of sodium hydroxide and 6.6 grams of sodium bicarbonate in distilled water. Of this buffer solution, 1568 grams is heated to boiling and 100 grams of a synthetic magnesium silicate absorptive clay having partial replacement of magnesium by lithium is added with high speed stifling. The mixture is maintained at 95° C. and stifling is continued until all the synthetic clay particles are dispersed. The paste is cooled to 25° C. and a solution of 32 grams of lithium hypochlorite in 300 grams of buffer solution is added. Finally, the pH is adjusted to 10.5 by the slow addition of sodium hydroxide while stirring. The resulting formulation is a nearly clear paste containing 5% synthetic clay and 0.5% by weight of available chlorine.

### Example 2

### Alternative Paste Formulation

A composition is prepared as described in Example 1, but using water in place of the buffer solution, and chloramine T in place of the lithium hypochlorite. The composition contains 0.5% by weight of available chlorine. Sodium hypochlorite and other sources of hypochlorite ion may be also be used.

### Example 3

### 0.1% HOCl Gel Formulation

An exemplary gel formulation is prepared by mixing a poly (acrylic acid) with an aqueous hypohalite solution. In this example, 250 ml of a 0.2% HOCl solution at pH 4.5 is made in accordance with U.S. Pat. No. 6,426,066, as described above. About 10 g of Carbopol 676 is used in this example, but other suitable polymers, e.g. Carbopol 974P, may also be used. The polymer is passed through a sieve or strainer to break up any clumps. The polymer is added slowly to 250 ml of purified water and the solution is mixed to promote the powder to dissolve evenly. The aqueous hypohalite solution (e.g. hypochlorite solution, as above) is then added to the polymer solution to result in a 0.1% HOCl gel formulation. Sodium hydroxide or hydrochloric acid may be used if desired to adjust the pH of the final formulation.

### Example 4

### 0.05% Gel Formulation

In this example, 250 ml of a 0.1% HOCl solution at pH of 7.5 is made by mixing reagent grade NaOCl in purified water and adjusting the pH using HCl (1N or more dilute solutions, as needed). About 10 g of Carbopol 974P is passed through a sieve or strainer to break up any clumps. The polymer is added slowly to 250 ml of purified water and the solution is mixed to promote the powder to dissolve evenly. The aqueous hypohalite solution is then added to the polymer solution to result in a 0.05% HOCl gel formulation. Sodium hydroxide or hydrochloric acid may be used if desired to adjust the pH of the final formulation.

### Example 5

### Skin Patch

A skin patch of the composition of the present application is prepared by conventional pharmaceutical methods. A square piece of sterile, finely woven gauze one centimeter on each side is placed in the center of a square piece of occlusive surgical adhesive tape two centimeters on each side. To the gauze is applied 0.4 mL of the gel of Example 3; the gel is allowed to soak into the gauze. This skin patch is prepared and can be stored for up to about 2 years before being applied to a subject to treat or prevent an infection of a nail, claw, or hoof.

### Examples 6-7

### Treatment of Onychomycosis with Gel Formulation

The gel formulations of Examples 3 and 4 are provided to human subjects having distal subungual onychomycosis. Each infected nail to be treated is examined, cultured, and photographed before treatment begins. Culturing is performed as described by B. Elewski (Journal of the American Academy of Dermatology, v. 35 (number 3, part 2): S6-S9, incorporated herein by reference): The nail to be sampled is first swabbed liberally with alcohol to eliminate as many bacteria as possible, as bacteria could overgrow and inhibit the growth of fungi. A small curette or special nail clipper is used to cut away the distal end of the nail plate; a curette is then used to scrape debris from the nail bed at a site as close to the cuticle as possible; scrapings from the under surface of the nail plate may be included. The shavings specimen is divided in half, each half being spread on a petri dish containing Sabouraud glucose agar. One petri dish is cycloheximide-free, while the other contains cycloheximide in the agar. Chloramphenicol is usually added to the culture media in both dishes to inhibit bacterial growth. The dishes are incubated for 7 to 14 days and then examined microscopically to identify any fungal or yeast growth. As cycloheximide inhibits nondermatophyte growth but not dermatophyte growth, colonies that appear on both dishes are likely to be dermatophytes, while those that only appear on the cycloheximide-free dish are likely to be nondermatophyte species.

Each of the subjects is instructed to topically apply the formulation to the affected nail or nails twice daily for eight weeks. The subjects return to the clinic every seven days, when each nail is again examined, cultured and photographed.

### Example 8

### Treatment of Onychomycosis with Patches

The skin patches of Example 5 are provided to subjects having onychomycosis on one or more toenails. Each infected nail to be treated is examined, cultured, and photographed before treatment begins. Culturing is performed as in Example 6. Each of the subjects is instructed to topically apply such a skin patch to the affected nail or nails, replacing the old patch with a new one every 24 to 48 hours. The subjects return to the clinic every seven days, when each nail is again examined, cultured and photographed.

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating or preventing an infection of a nail, claw, or hoof comprising administering a pharmaceutically acceptable formulation comprising hypohalite and a formulating agent to a subject in need thereof.

2. The method of claim 1, wherein the infection is a fungal infection.

3. The method of claim 1, wherein the hypohalite is hypochlorite.

4. The method of claim 1, wherein the formulating agent comprises a water-swellable polymer.

5. The method of claim 4, wherein the water-swellable polymer is a poly (ethylene oxide).

6. The method of claim 4, wherein the water-swellable polymer is a poly (acrylic acid).

7. The method of claim 1, wherein the formulating agent comprises a water-absorptive clay.

8. The method of claim 7, wherein the water-absorptive clay is bentonite.

9. The method of claim 1, wherein the formulation has a pH from about 3.5 to about 8.

10. A method of treating or preventing an infection of a nail, claw, or hoof comprising administering to a subject in need thereof a composition comprising:
a first active agent comprising hypohalite;
a second active agent, and
a formulating agent;
wherein the second active agent is an antiinfective agent of a different class than the first active agent.

11. The method of claim 10, wherein the second active agent is an N-halogenated or N,N-dihalogenated amine compound.

12. The method of claim 11, wherein the N-halogenated or N,N-dihalogenated amine compound is 2-(dichloroamino)-2-methylpropane-1-sulfonic acid, 2-(chloroamino)-2-methylpropane-1-sulfonic, or N-chlorotaurine.

13. The method of claim 10, wherein the second active agent is selected from the group consisting of allylamines, triazoles, imidazoles, amorolfine, ciclopirox, alamine, sodium pyrithione, bifonazole, propylene glycol, urea, lactic acid, benzyl alcohol, phenoxyethanol, phenethylalcohol, iodopropyl butyl carbamate, paraben, quaternary ammonium compounds, benzoyl peroxide and chlorhexidine.

14. The method of claim 1, wherein the pharmaceutical composition further comprises a carrier, excipient, film-forming agent, humectant, penetration enhancer, plasticizer, solvent, co-solvent, plasticizer, or surfactant.

15. A bandage for the treatment or prevention of an infection of a nail, claw, or hoof, comprising a nail-facing layer wherein the nail-facing layer contains a hypohalite formulation.

16. The bandage of claim 15, wherein the hypohalite formulation comprises hypochlorite and a water-swellable polymer.

17. The bandage of claim 15, wherein the nail-facing layer allows for release of hypohalite over about 24 to about 48 hours.

18. A method of treating or preventing a skin infection comprising administering a pharmaceutically acceptable formulation comprising hypohalite and a formulating agent to a subject in need thereof.

19. The method of claim 18, wherein the hypohalite is hypochlorite.

20. The method of claim 18, wherein the formulating agent comprises a water-swellable polymer.

21. The method of claim 18, wherein the formulation has a pH from about 3.5 to about 8.
